(19) United States
(12) Patent Application Publication
(10) Pub. No.: US 2018/0243140 A1
(43) Pub. Date: Aug. 30, 2018

(54) ANTIMICROBIAL ELASTIC SUPPORT BANDAGES

(71) Applicant: SILVER X TECHNOLOGIES, Phoenix, AZ (US)

(72) Inventors: Edward Song, Phoenix, AZ (US); Michael Wilmink, Phoenix, AZ (US)

(21) Appl. No.: 15/906,649

(22) Filed: Feb. 27, 2018

Related U.S. Application Data
(60) Provisional application No. 62/464,905, filed on Feb. 28, 2017.

(30) Foreign Application Priority Data
Jul. 11, 2017 (CN) 201710563392.6

(51) Int. Cl. A61F 13/00; A61F 13/02; A61F 15/00; A61L 15/14; A61L 15/08; A61L 15/12; A61L 15/10

(52) U.S. Cl. CPC A61F 13/00063; A61F 13/00012; A61F 13/00017; A61F 13/0233; A61F 15/006; A61L 15/14; A61L 2300/404; A61L 15/12; A61L 15/10; A61F 2013/00238; A61F 2013/0028; A61F 2013/00234; A61L 15/08

(57) ABSTRACT

Disclosed herein are antimicrobial elastic support bandages including a plurality of warp threads extending along at least a portion of the length thereof and including non-antimicrobial warp regions and antimicrobial warp regions arranged in an alternating fashion across the width thereof, wherein (a) a non-antimicrobial warp region comprises an elastic material that is stretchable in the direction of the length, (b) an antimicrobial warp region comprises (i) a flexible fiber coupled to an antimicrobial metal, and (ii) an elastic material that is stretchable in the direction of the length, wherein the flexible fiber is coupled to the elastic material, and (c) at least a portion of the warp threads are separated by about 3 microns to about 20 microns; and further including a plurality of weft threads extending across at least a portion of the width and comprising a biocompatible material, wherein at least some of the weft threads are separated by about 3 microns to about 20 microns, wherein at least a portion of the warp threads are interwoven with at least a portion of the weft threads.

### CROSS-REFERENCE TO RELATED APPLICATION

This application claims the benefit under 35 U.S.C. § 119(e) to U.S. Provisional Application No. 62/464,905, filed Feb. 28, 2017, and claims the beneft under 35 U.S.C. § 119(d) to Chinese Patent Application No. 201710563392.6, filed Jul. 11, 2017, which applications are hereby incorporated by reference in their entireties.

### BACKGROUND

This disclosure relates to antimicrobial bandages, including antimicrobial elastic support bandages, which provide cover, support, or compression to an injured portion of a body of a subject or patient while having antimicrobial activity.

Support bandages, such as compression wraps and short-stretch wraps, are generally made from a woven combination of elastic and inelastic textile materials. The elastic material allows the support bandage to stretch partially or fully across an injured body part, in some cases wrapping around the injured body part to provide support and compression thereto, while the inelastic material is interwoven with the elastic material and covers the injured body part. U.S. Pat. Nos 6,267,744, 4,207,885 and 5,647,842 describe stretchable bandages with various weave patterns for achieving desired characteristics of stretch and support.

In many instances, support bandages are used to alleviate slow-healing injuries, such as ligament sprains, muscular or tendon strains, or vascular ulcers or edema. However, prolonged use of a single support bandage can invite microbial growth on, in, or underneath the bandage, increasing the risk of infection to the subject or to those in close proximity to the subject, such as family members or care providers. Further, microbial growth can be accompanied by unpleasant odors and unsightly discoloration of the support bandage. Yet, discarding and replacing support bandages after a single or a few uses is costly and time-consuming, and conventional support bandages can lose their compression strength and elasticity after repeated wash and use cycles. Thus, there is a need in the art for support bandages that possess antimicrobial properties, provide appropriate coverage and support to injuries, and are designed for long-term use.

### BRIEF SUMMARY

Described herein are antimicrobial elastic support bandages. A specific embodiment provides an antimicrobial elastic support bandage having a length and a width and including a plurality of warp threads extending along at least a portion of the length and including non-antimicrobial warp regions and antimicrobial warp regions arranged in an alternating fashion across the width, wherein (a) a non-antimicrobial warp region comprises an elastic material that is stretchable in the direction of the length, (b) an antimicrobial warp region comprises (i) an antimicrobial metal coupled to a flexible fiber, and (ii) an elastic material that is stretchable in the direction of the length, wherein the flexible fiber is coupled to the elastic material, and (c) at least a portion of the warp threads are separated by about 3 microns to about 20 microns; and further including a plurality of weft threads extending across at least a portion of the width and comprising a biocompatible material, wherein at least some of the weft threads are separated by about 3 microns to about 20 microns, wherein at least a portion of the warp threads are interwoven with at least a portion of the weft threads.

### BRIEF DESCRIPTION OF THE FIGURES

FIG. 1 shows a top view of a portion of one embodiment of an antimicrobial elastic support bandage according to the present disclosure.

### DETAILED DESCRIPTION

Various embodiments of the present disclosure provide antimicrobial elastic support bandages comprised of a woven pattern of warp and weft threads. The warp threads comprise regions of non-antimicrobial warp threads and antimicrobial warp threads. An antimicrobial warp region includes an elastic material, a flexible fiber, and an antimicrobial metal, and a non-antimicrobial warp region comprises an elastic material. The weft threads include a biocompatible material.

Prior to setting forth this disclosure in more detail, it may be helpful to have an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives or enumerated components. As used herein, the terms “include,” “have” and “comprise” are used synonymously, which terms and variants thereof are intended to be construed as non-limiting.

As used herein, “antimicrobial” refers to one or more properties of a substance or composition of matter (e.g., of the antimicrobial metals discussed herein) to kill and/or to reduce, slow, or prevent the growth of a microorganism such as a bacteria, yeast, algae, or fungus, especially a pathogenic microorganism. Conversely, the term “non-antimicrobial” refers to a substance or composition of matter that is not inherently capable of killing and/or reducing, slowing, or preventing the growth of a microorganism such as a bacteria, yeast, or fungus.

As used herein, a “support bandage” refers to a piece or strip of flexible textile material used to support and/or compress an injured part of the body of a subject. A support bandage according to the present disclosure may cover, protect, bind, immobilize, or restrict the motion of the injured body part in providing the support and/or compression. In the description that follows, the terms “antimicrobial elastic support bandage,” “elastic support bandage,” and “antimicrobial support bandage” are used interchangeably to refer to antimicrobial elastic support bandages according to the present disclosure, unless clearly otherwise intended.

An “elastic material”, as used herein, is a material having elasticity (i.e., an object made of an elastic material it returns to its original shape following application of a force load and resulting deformation) and being suitable for use in a threaded or fibrous form. Non-limiting examples of elastic materials for use in the presently disclosed support bandages include the polyester-polyurethane copolymer fiber, i.e., elastane fiber or Spandex fiber, (under various tradenames as Lycra™, Elaspan, Acepora, Creora, Inviya, Roica, Dolastan, Linel, and ESPA), vinyl, nylon, rubber latex, and elastin, which is a highly elastic protein found in connective tissue in vertebrates. Elasticity is can be quantified using Young's modulus (also called the elastic modulus), which is essentially the ratio of stress to strain along a given axis. Young's modulus enables the quantification, measured in units of pressure, of the change in dimension of a bar of a given material under tensile or compressive strength, and is calculated by dividing the tensile stress by the extensional strain in the elastic portion of the physical stress-strain curve:

$$E ≡ \frac{σ ( ɛ )}{ɛ} = \frac{F \text{/} {A}_{0}}{Δ L \text{/} {L}_{0}} = \frac{{FL}_{0}}{{A}_{0} Δ L}$$

where E is the Young's modulus, F is the force exerted on the object under tension, A₀ is the cross-sectional area to which the force is applied, ΔL is the amount of length change of the object, and L₀ is the original length of the object. Materials with a low Young's modulus have a higher degree of elasticity. For example, small strain rubber has an approximate Young's modulus of 0.01-0.1 GPa (1.45-14.5×10⁻³ Mpsi), while antimicrobial metals measure much higher (silver=83 GPa; bronze=96-120 GPA; copper=117 GPa; molybdenum=329-330 GPa).

The terms “warp thread” (or “warp”) and “weft thread” (or “weft”) refer, respectively, to threads that define the length and width of a two-dimensional woven fabric, such as the fabric comprising an elastic support bandage. Generally, warp threads run in parallel fashion along the length of a woven fabric, while weft threads run orthogonally or transversely to the direction of the warp threads and in parallel fashion to one another to define the width of the woven fabric. The warp and weft threads are interwoven to define the woven fabric. Various methods of weaving warp and weft threads are known in the art (see, e.g., U.S. Pat. Nos 6,267,744, 4,207,885 and 5,647,842). An antimicrobial elastic support bandage according to the present disclosure may include a single weft thread that doubles back on itself at each outer edge of the width of the bandage, or may include multiple weft threads. At either lengthwise or widthwise edge, the warp or weft threads may be bound, sealed, or otherwise coupled together to define a consistent closed edge of the bandage.

An “antimicrobial metal” refers to a metal or metal alloy that possesses antimicrobial properties. Non-limiting examples of antimicrobial metals include silver, gold, copper, iron, zinc, aluminum, cobalt, nickel, zirconium, molybdenum, bismuth, and alloys and other combinations thereof. Non-limiting examples of alloys include bronze (an alloy of copper and tin), brass (an alloy of copper and zinc), cupronickel (an alloy of copper and nickel), copper-nickel-zinc, Argentium sterling silver (silver-copper-germanium), Billon, Britannia silver (silver-copper), Dore' bullion (silver-gold), Electrum (silver-gold), Goloid (silver-copper-gold), Platinum sterling (silver platinum), Shibuichi (silver-copper), sterling silver (silver-copper), and Tibetan silver (silver-copper). Without wishing to be bound by theory, it is believed that cations released by antimicrobial metals bind to negatively charged surfaces on microorganisms, thereby entering the intracellular environment to cause DNA damage and oxidative stress to the microorganism, which in turn lead to cell death.

The term “coupled to” refers to chemical or mechanical attachment of a first material or component to a second material or component. For example, as will be discussed further herein below, an antimicrobial elastic support bandage of the present disclosure includes at least one region of warp thread(s) including an antimicrobial metal coupled to a flexible fiber; the antimicrobial metal may be chemically bonded to the flexible fiber using linker chemistry, bonded to the flexible fiber using an adhesive substance, or mechanically coupled to the flexible fiber by interweaving with the fiber or tied to the flexible fiber by one or more other threads or fibers. In certain embodiments, the antimicrobial metal coats (i.e., covers) at least a portion of an outer surface of the flexible fiber. In certain embodiments, the antimicrobial metal is impregnated within at least a portion of the flexible fiber (e.g., the flexible fiber may be formed, cut, or woven fully or partially around the antimicrobial metal, or the flexible fiber may receive the antimicrobial metal therein by mechanical force, chemical reaction, or another means). In certain embodiments, the antimicrobial metal is coated on or interwoven with at least a portion of the flexible fiber. Composite threads comprised of flexible fibers and an antimicrobial metal are commercially available from Noble Biomaterials (Scranton, Pa., USA).

The term “biocompatible material” refers to a material that is not harmful to living tissue of a human or domestic animal and is useful in a woven textile product such as a bandage. Non-limiting examples include cotton, hemp, nylon, rayon, and polyester, though many other such materials are known and are appropriate for use with the presently disclosed support bandages. In preferred embodiments, the biocompatible material is a material that allows for air and moisture exchange between the surface of the bandaged body region and the local environment (i.e., the biocompatible material “breathes well”). In other preferred embodiments, the biocompatible material is derived from natural fiber such as cotton.

As used herein, a “short stretch bandage” is an elastic bandage that stretches along its length and, optionally, its width, to cover and optionally provide compression to a wound or an injured area, but does not wrap completely around the injured body part or area. A “compression wrap”, as used herein, is an elastic bandage that wraps around a body part or portion to cover and provide compression to a wound or an injured area. Generally, short stretch bandages have lower lock-out points (the point of extension at which the structure of the bandage prevents further extension with increasing load when it is under tensional load) as compared to compression wraps. Other characteristics of short stretch bandages and compression wraps are described herein.

In one aspect, an antimicrobial elastic support bandage has a length and a width and comprises a plurality of warp threads extending along at least a portion of the length and comprising non-antimicrobial warp regions and antimicrobial warp regions arranged in an alternating fashion across the width, wherein (a) a non-antimicrobial warp region comprises an elastic material that is stretchable in the direction of the length, (b) an antimicrobial warp region comprises (i) an antimicrobial metal coupled to a flexible fiber, and (ii) an elastic material that is stretchable in the direction of the length, wherein the flexible fiber is coupled to the elastic material, and (c) at least a portion of the warp threads are separated by about 3 microns to about 20 microns; and further comprises a plurality of weft threads extending across at least a portion of the width and comprising a biocompatible material, wherein at least some of the weft threads are separated by about 3 microns to about 20 microns, wherein at least a portion of the warp threads are interwoven with at least a portion of the weft threads. In certain preferred embodiments, each of the weft threads is separated from adjacent weft threads by about 3 to 20 microns, and each of the warp threads is separated from adjacent warp threads by about 3 to 20 microns in order to allow for even passage of air, light, and moisture across the area of the elastic support bandage.

In certain embodiments, the antimicrobial metal comprises silver, gold, copper, iron, zinc, bismuth, aluminum, cobalt, nickel, zirconium, molybdenum, or any combination or alloy thereof. In certain preferred embodiments, the antimicrobial metal comprises silver or an alloy thereof. The antimicrobial metal and the flexible fiber may be coupled by any suitable chemical or mechanical means. In certain embodiments, the antimicrobial metal coats at least a portion of an outer surface of the flexible fiber. In certain embodiments, the antimicrobial metal is impregnated within at least a portion of the flexible fiber. In certain embodiments, the antimicrobial metal is interwoven with at least a portion of the flexible fiber.

The amount of the antimicrobial metal in a given antimicrobial warp region and in the elastic support bandage as a whole affects not only the antimicrobial properties, but also the weight, strength, and stretching properties of the elastic support bandage. Therefore, in certain embodiments, the elastic support bandage comprises a sufficient amount of an antimicrobial metal or alloy thereof to provide antimicrobial properties to the bandage without undesirably limiting the elasticity or increasing the weight of the bandage. In certain embodiments, the antimicrobial metal comprises about 1%, about 2%, about 3%, about 4%, about 5%, about 6%, about 7%, about 8%, about 9%, about 10%, about 15%, or about 20% of the woven antimicrobial support bandage by weight.

It will be understood that in order for the antimicrobial elastic support bandage to have the desired ability to stretch in order to cover a wound and, in some cases, to wrap around the injured body part in order to provide support, both the non-antimicrobial warp regions and the antimicrobial warp regions include an elastic material. In certain preferred embodiments, the elastic material comprises elastane (also known as spandex and by various commercial names as described previously). The coupled antimicrobial metal and flexible fiber are in turn coupled to a thread of an elastic material (e.g., adhered together, chemically bonded together, twisted together, or held together by other threads) and therefore expand and contract along with the elastic material. Because the antimicrobial metal is inelastic, it will be understood that the antimicrobial warp regions of the elastic support bandage could have a somewhat lesser elasticity as compared to the non-antimicrobial warp regions.

It will be understood that either or both of a non-antimicrobial region and an antimicrobial region may comprise a single warp thread or one or more adjacent warp threads, and that the alternating array of antimicrobial regions and non-antimicrobial regions may take any form. For example, in certain embodiments, an elastic support bandage comprises only a single antimicrobial region, with non-antimicrobial regions on either side of the antimicrobial region. In certain other embodiments, an elastic support bandage has two or more antimicrobial regions, with non-antimicrobial regions interspersed between and, optionally, flanking the antimicrobial regions. In addition, in certain embodiments one or more edge of the bandage (i.e., a lengthwise edge or a widthwise edge) may be of a different material or arrangement than the warp and weft threads. That is, the woven fabric of the elastic support bandage may be capped or bounded along one or more edge by a different, optionally non-woven, material in order to, for example, preserve the integrity of the woven pattern over time or form a softer or smoother or more yielding contact edge with the skin of the subject.

In certain embodiments, a binding agent may be used to add strength and structure to the antimicrobial elastic support bandage. The binding material may be used to coat (e.g., by spraying, rolling, or dipping) the support bandage, or it may be used to cross-link different components of the support bandage, such as the elastic material and the antimicrobial metal/flexible fiber.

Additional features may be employed to improve the feel and/or appearance of the elastic support bandage. For example, in certain embodiments at least one of the non-antimicrobial warp regions and/or at least one of the antimicrobial warp region further comprises a second material coupled to the elastic material to, for example, provide additional padding, comfort, or aesthetic qualities to the elastic support bandage. The second material may or may not have elasticity, or at least has less elasticity than the elastic material. Typically the second material may be polyester or cotton fiber. Thus, in certain embodiments, an antimicrobial elastic support bandage comprises one or more polyester threads of a color that matches, individually or in the aggregate, the skin tone of the subject. In an exemplary embodiment, non-antimicrobial warp regions include white polyester or red polyester such that the overall effect is to provide a pinkish flesh tone color to the bandage. Other desired tones or colors can be achieved using different colors and/or arrangements of colors in the warp and/or weft threads. Polyester threads, typically of 150 deniers (or 150D), may be twisted or otherwise coupled with Spandex to form individual warp thread to improve elasticity.

While an elastic support bandage according to the present disclosure is stretchable along its length in order to cover and/or wrap around the injured body part, and to return to about its relaxed length when not under tension, the bandage may, but need not be stretchable across its width. Thus, in certain embodiments, a weft yarn consists of one or more non-elastic biocompatible material. In certain other embodiments, a weft yarn comprises a non-elastic biocompatible material (e.g., cotton) coupled to at least one elastic material.

Antimicrobial elastic support bandages according to the present disclosure may be used to provide protection and compression to injuries of different sizes and on different parts of the body. Briefly, support bandages apply pressure as determined by the elasticity or “extensibility” of the bandage material and the tightness and tension of material as applied. Appropriate administration and use of support bandages can improve circulation and reduce edema (accumulation of fluid in extra-vascular tissue) by reinforcing reabsorption of capillary fluid into the veins and lymph vessels and/or by decreasing venous and arterial blood volume, and thereby accelerating blood flow, in the extremities. Managing appropriate blood, lymph, and solute levels (e.g., proteins) by use of support bandages and compression mitigates or prevents unwanted blood pooling and the excessive accumulation of white blood cells at sites of injury. As noted above, support bandages may be “short-stretch” or “long-stretch”, depending on their properties of extension. Short-stretch bandages of the present disclosure preferably lock-out at up to about 70% extension, more preferably at about 30% to about 40% extension. However, it will be understood that a short-stretch bandage within the scope of the present disclosure may lock-out at about 10%, about 20%, about 30%, about 40%, about 50%, about 60%, about 70%, about 80%, or about 90% extension. Long-stretch bandages of the present disclosure may lock-out at about 100%, 110%, 120%, 130%, 140%, 150%, 160%, 170%, 180%, 190%, or 200% or greater, and preferably at about 140% extension or more.

The amount of pressure produced by a support bandage at a predetermined amount of extension is known as the “power” of the bandage. Two widely used standards for measuring support bandage power are the so-called British and German standards. The British standard groups bandages into six categories:
Type 1 (retention, light compression elastic bandages); Type 2 (inelastic, light-compression short-stretch bandages); Type 3A (light-compression, elastic, long-stretch bandages; up to 20 mmHg); Type 3B (light-compression, elastic, long-stretch bandages; 21-30 mmHg); Type 3C (moderate-compression bandages; 31-40 mmHg); Type 3D (high-compression bandages; 41-60 mmHg). The German standard provides four categories: Group 1 (light compression, 18.4-21.2 mmHg); Group 2 (light compression; 25.4-32.1 mmHg); Group 3 (moderate compression; 36.4-46.5 mmHg); and Group 4 (46. 5 mmHg-59 mmHg).

The inventors of the presently disclosed subject matter have determined that antimicrobial protection may be conferred upon a support bandage while maintaining desirable characteristics of extensibility, weight, and bandage power within the above standards by combining antimicrobial metal with a flexible fiber and elastic materials to form warp regions along the length of the support bandage. Advantageously, the antimicrobial properties are maintained over multiple use-and-wash cycles, and the inclusion of inelastic antimicrobial metal increases the durability of the support bandage.

Depending on the nature of the injury and body proportions of the injured subject, elastic support bandages of different lengths and stretch properties may be desirable. For example, in certain situations, a short stretch bandage may be more suitable for protecting and providing support to the injury. Accordingly, in certain embodiments, the antimicrobial elastic support bandage is a short stretch bandage. In certain embodiments, a short stretch bandage of the present disclosure locks out at up to about 70% extension, and preferably locks out at from about 30 to 40% extension. In other situations, a compression wrap may be preferred. Thus, in alternate embodiments, the antimicrobial elastic support bandage is a compression wrap. In certain embodiments, a compression bandage of the present disclosure locks out at at least about 70% extension, and preferably locks out at at least about 140% extension.

In certain embodiments, an elastic support bandage according to the present disclosure further comprises a self-securing mechanism proximal to an end of the length. The self-securing mechanism attaches to another portion of the elastic support bandage and can keep the elastic support bandage securely on or around the injured body part, applying consistent protection and compression without requiring further attention from the subject. In certain embodiments, the self-securing mechanism is selected from the group consisting of: a hook and fastener; a button and fastener; a loop and fastener; an adhesive portion; Velcro; a clip, or any combination thereof.

### EXAMPLES

### Example 1

### Making an Antimicrobial Elastic Support Bandage

Silver-coupled flexible fiber (“X-Static™”) was obtained from Noble Biomaterials (Scranton, Pa., USA). The X-Static™ fiber was twisted along with 100% spandex yarn (Model Number 150D; Type: Spandex Yarn; Style: Bare Yarn; Evenness: High Uniformity; Strength: High Tenacity, Model Number: 150D, Twist: Untwisted; Pattern: Raw; Yarn Count: High Elastic) to form antimicrobial warp threads. To form the non-antimicrobial warp threads, red and white polyester (100%; Yarn Count: 150D; Pattern: Twisted Textured Yarn; Yarn Type: DTY; Filament; Texturized; Twist: 120TPM; Evenness: Equal) threads were twisted with 100% spandex yarn as described above. The antimicrobial and non-antimicrobial warp threads were arranged in an alternating pattern of: white polyester+spandex; red polyester+spandex; and X-Static™+spandex at 2%, 3%, 5%, or 10% X-static™ by weight. Three white polyester+spandex threads were used to visually define the lengthwise edges of the bandage. Cotton (100%; Yarn Count 10 s; Twist: <450 tpm; Strength: 250-350 cn; Pattern: Raw; Evenness: cv %1.5) threads were used to form the weft threads. The warp and weft threads were interwoven to form the antimicrobial elastic support bandage, allowing spaces of about 3 microns between adjacent threads. At the bottom surface of one end of the bandage, a Velcro strip was attached by sewing.

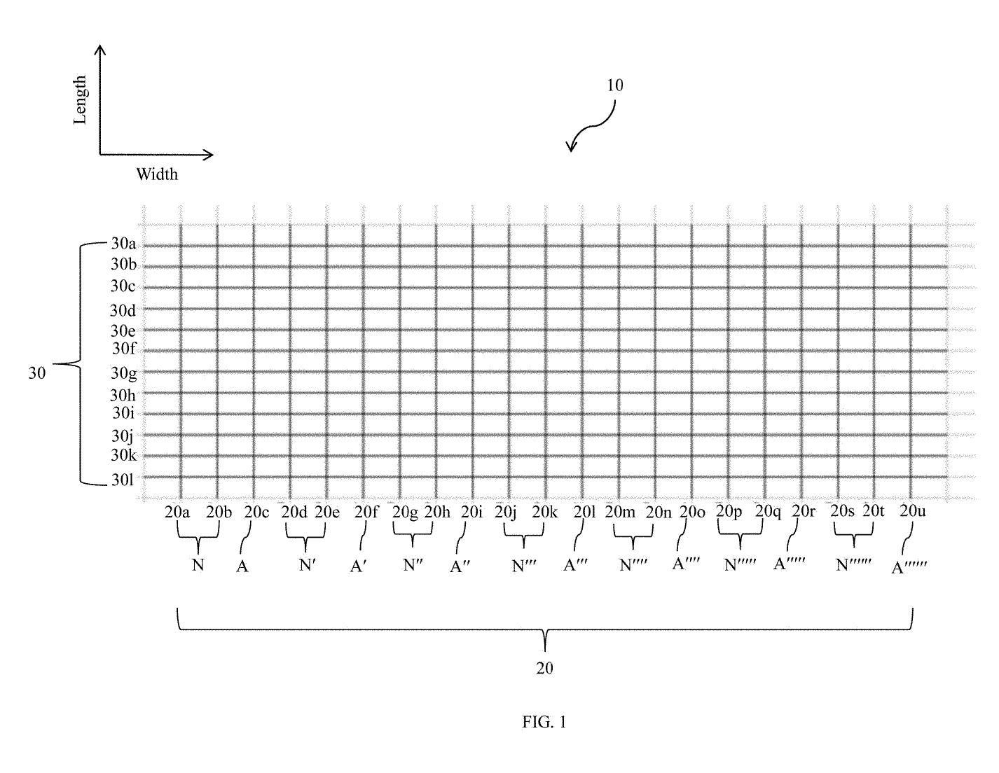

In one embodiment, shown in FIG. 1, the antimicrobial elastic support bandage 10 was formed as described above to include a plurality 20 of warp threads 20a-20u extending along a portion of the length and comprising non-antimicrobial warp regions N-N′″″ and antimicrobial warp regions A-A′″″ arranged in an alternating fashion across the width. The non-antimicrobial warp regions N-N′″″ are comprised of spandex (not shown) twisted with red or white polyester (not shown). The antimicrobial warp regions A-A are comprised of X-Static™ threads (e.g., 20c, 20f, 20i, 20l, 20o, 20r, 20u) twisted with spandex. Three non-antimicrobial warp threads comprising white polyester twisted with spandex (not shown) form the lengthwise edges of the elastic support bandage. The weft threads are cotton (30a-30l). Each warp and weft thread is separated from adjacent threads by about 3 microns. In one example, a final product at 5% wt X-Static™ fabric is further comprised of 50% wt cotton, 25% wt polyester, and 20% wt spandex. In another example, a final product at 10% wt X-Static™ fabric is further comprised of 47% wt cotton, 23% wt polyester, and 20% wt spandex.

### Example 2

### Antimicrobial Activity of the Elastic Support Bandage

Antimicrobial activity of the elastic support bandages described in Example 1 (2% wt, 3% wt, 5% wt, 10% wt X-Static™) was assessed according to a standard ISO 20743: 2013(E) Absorption MRSA for four hours (The MicroStar Lab, Crystal Lake, Ill.). Briefly, the elastic support bandages were inoculated with bacteria and allowed to culture under for four hours, after which bacterial colonies were counted.

More specifically, sample replicates were placed into sterile containers and inoculated with the test organism. Three replicates of the test and control samples were tested immediately after inoculation (Time=0) and after the contact time. All inoculated test items were incubated for the contact time at 35° C. At each contact time, 20 mL of neutralizing broth was added to each container and shaken thoroughly to facilitate the release of the inoculum from the sample into the neutralizing broth. Serial dilutions of the neutralizing broth containing the inoculum were plated. All plates were incubated at 35° C. for 48 hours. After incubation, bacterial colonies were counted and recorded. Results are reported as Log₁₀. Test conditions and variables are described in Table 1:

TABLE 1
Test Variables for ISO Antimicrobial Activity

| | |
|---|---|
| Test Organism | Methicillin-resistant |
| | S. aureus (MRSA) ATCC 33591 |
| Dilution Medium Used | Tryptic Soy Broth diluted |
| | 20X with 0.05% Triton X-100 |
| Starting Concentration | 3.7 × 10⁵ CFU/mL |
| Sample Size | 0.8 gram cut from larger |
| | submitted pieces |
| Untreated Control | Sterile filter paper cut into |
| | 3.8 cm discs. Four (4) discs |
| | weighed 0.4 grams. |
| Number of Replicates per Sample | 3 |
| Amount of Inoculum per Replicate | 0.4 mL |
| Incubation Time of Samples | 4 hours |
| Neutralizing Broth Used | D/E Neutralizing Broth |
| Amount of Neutralizing Broth | 20 mL |
| Dilution Medium | Phosphate Buffer Saline |
| Plating Medium | Standard Methods Agar |
| Method of Sterilization/ | No |
| Pre-Cleaning | |
| Deviations from Standard | Standard sample size |
| | is 0.4 gram |
| | Standard inoculum amount per |
| | replicate is 0.2 mL |
| | Standard contact time is 18-24 hours |
| | Standard organisms are |
| | K. pneumoniae ATCC 4352 and |
| | S. aureus ATCC 6538 |

### Results

Results can be found in the data table below. The results pertain only to the sample tested. The differences in extremes for the control sample immediately after inoculation and after incubation were <1 log as required by the standard. The differences in extremes for the test samples immediately after inoculation and after incubation were <2 log as required by the standard. The growth value of the control samples (F) is calculated by subtracting the average common logarithm for the number of bacteria recovered from the control immediately after inoculation (C₀) from the average common logarithm for the number of bacteria recovered on the same control after the contact time (Cₜ). The growth value (F) of the control sample is expected to increase ≥1 log. The growth value of the control fabric for this test was 0.65. The equation for the growth value of the control sample (F) is as follows: F=Cₜ−C₀

Growth Value for Control

| | C₀ - Number | Cₜ- Number | Growth |
|---|---|---|---|
| | of Bacteria | of Bacteria | Value of |
| | Recovered | Recovered | Control (F) |
| Sample | Time = 0 | Time = 4 hours | F = Cₜ − C₀ |
| Control Sample | 5.12 | 5.77 | 0.65 |

The growth value of the test samples (G) is calculated in the same manner as the control growth value using the following equation: G=Tₜ−T₀

Growth Value for Test Samples

| | T₀ - Number | Tₜ- Number | Growth |
|---|---|---|---|
| | of Bacteria | of Bacteria | Value of |
| | Recovered | Recovered | Sample (G) |
| Sample | Time = 0 | Time = 4 hours | F = Tₜ − T₀ |
| 7487 DKCC | 5.12 | 3.11 | −2.01 |
| International 5X wash | | | |

The antibacterial activity value (A) is calculated by subtracting the growth value of a treated sample (G) from the growth value of the control (F). The calculation is as follows: A−F−G

Antibacterial Activity Value

| | | Growth | |
|---|---|---|---|
| | Growth | Value of | Antibacterial |
| | Value of | Test | Activity |
| Sample | Control (F) | Sample (G) | Value (A) |
| 7487 DKCC | 0.65 | −2.01 | 2.66 |
| International 5X wash | | | |

The efficacy of antibacterial property of the test specimen can be considered as follows per ISO 20743:2013 Annex F:

| Efficacy of | Antibacterial |
|---|---|
| Antibacterial Property | Value: A |
| Significant | 2 ≤ A ≤ 3 |
| Strong | A ≤ 3 |

Percent and Log Reduction

| | Average Number of | | |
|---|---|---|---|
| | Bacteria Recovered | Percent | Log |
| Sample | (CFU/sample) | Reduction | Reduction |
| Filter Paper Control | 5.9 × 10⁵ | | |
| 7487 DKCC | 1.3 × 10³ | 99.8% | 2.66 |
| International 5X wash | | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Patent Application No. 62/464,905 filed Feb. 28, 2017 and Chinese Patent Application No.: 201710563392.6 filed Jul. 12, 2017, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antimicrobial elastic support bandage having a length and a width, comprising:
a plurality of warp threads extending along the length and comprising non-antimicrobial warp regions and antimicrobial warp regions arranged in an alternating fashion across the width, wherein
(a) each non-antimicrobial warp region comprises one or more warp threads of a first elastic material that is stretchable in the direction of the length,
(b) each antimicrobial warp region comprises at least one warp thread comprising (i) a flexible fiber coupled to an antimicrobial metal , and (ii) a second elastic material that is stretchable in the direction of the length, wherein the flexible fiber is further coupled to the second elastic material, and
(c) two adjacent warp threads are separated by about 3 microns to about 20 microns; and
a plurality of weft threads extending across the width and comprising a biocompatible material, wherein at least some of the weft threads are separated by about 1 microns to about 20 microns,
wherein the warp threads are interwoven with the weft threads.

2. The antimicrobial elastic support bandage of claim 1, wherein the antimicrobial metal comprises silver, gold, copper, iron, zinc, bismuth, aluminum, cobalt, nickel, zirconium, molybdenum, or any combination or alloy thereof.

3. The antimicrobial elastic support bandage of claim 2, wherein the antimicrobial metal comprises silver.

4. The antimicrobial elastic support bandage of claim 1, wherein the antimicrobial metal comprises an alloy.

5. The antimicrobial elastic support bandage of claim 1, wherein the antimicrobial metal coats at least a portion of an outer surface of the flexible fiber.

6. The antimicrobial elastic support bandage of claim 1, wherein the antimicrobial metal is impregnated within at least a portion of the flexible fiber.

7. The antimicrobial elastic support bandage of claim 1, wherein the antimicrobial metal is interwoven with at least a portion of the flexible fiber.

8. The antimicrobial elastic support bandage of claim 1, wherein the flexible fiber is interwoven with the second elastic material.

9. The antimicrobial elastic support bandage of claim 1, wherein the first elastic material and the second elastic material comprises elastane fiber.

10. The antimicrobial elastic support bandage of claim 1, wherein the non-antimicrobial region and/or antimicrobial region further comprises weft threads made of fibers of a second material.

11. The antimicrobial elastic support bandage of claim 10 wherein the second material comprises polyester.

12. The antimicrobial elastic support bandage of claim 1, wherein the biocompatible material comprises cotton.

13. The antimicrobial elastic support bandage of claim 1, wherein the flexible fiber coupled to the antimicrobial metal comprises about 1%-10% of the woven antimicrobial support bandage by weight.

14. The antimicrobial elastic support bandage of claim 13, wherein the flexible fiber coupled to the antimicrobial metal comprises about 5% of the woven antimicrobial support bandage by weight.

15. The antimicrobial elastic support bandage of claim 1, wherein the woven antimicrobial support bandage is a short stretch bandage or a compression wrap.

16. The antimicrobial elastic support bandage of claim 15, wherein the bandage locks out at up to about 70% extension.

17. The antimicrobial elastic support bandage of claim 16, wherein the bandage is a short stretch bandage and locks out at from about 30 to 40% extension.

18. The antimicrobial elastic support bandage of claim 15, wherein the bandage is a compression wrap and locks out at least about 140% extension.

19. The antimicrobial elastic support bandage of claim 15, further comprising a self-securing mechanism proximal to an end of the length.

20. The antimicrobial elastic support bandage of claim 19, wherein the self-securing mechanism is selected from the group consisting of: a hook and fastener; a button and fastener; a loop and fastener; an adhesive portion; Velcro; a clip, or any combination thereof.
